(19) United States
(12) Patent Application Publication
(10) Pub. No.: US 2024/0257903 A1
(43) Pub. Date: Aug. 1, 2024

(54) METHOD AND APPARATUS FOR DETERMINING ANTIGENIC SPECIFICITY

(71) Applicant: TENCENT TECHNOLOGY (SHENZHEN) COMPANY LIMITED, Shenzhen (CN)

(72) Inventors: Yu ZHAO, Shenzhen (CN); Xiaona SU, Shenzhen (CN); Jianhua YAO, Shenzhen (CN); Bing HE, Shenzhen (CN); Zhimeng XU, Shenzhen (CN)

(21) Appl. No.: 18/629,287

(22) Filed: Apr. 8, 2024

Related U.S. Application Data
(63) Continuation of application No. PCT/CN2023/118539, filed on Sep. 13, 2023.

(30) Foreign Application Priority Data
Oct. 12, 2022 (CN) 202211247236.6

(51) Int. Cl. G16B 15/30; G16B 30/00; G16B 40/20

(52) U.S. Cl. CPC G16B 15/30; G16B 30/00; G16B 40/20

(57) ABSTRACT

Embodiments of this application provide a method and apparatus for determining antigenic specificity. The method includes acquiring double-stranded biological information of a cell receptor; performing word coding processing on the double-stranded biological information to obtain an amino acid word sequence, the amino acid word sequence comprising an amino acid word representation; performing feature extraction on the cell receptor based on the amino acid word sequence with a pre-trained amino acid sequence prediction model to obtain an amino acid sequence representation of the cell receptor, the amino acid prediction model being trained with masked sample data; and determining the antigenic specificity of the cell receptor based on the amino acid sequence representations.

### RELATED APPLICATIONS

This application is a continuation of PCT Application No. PCT/CN2023/118539, filed on Sep. 13, 2023, which claims priority of Chinese Patent Application No. 202211247236.6, filed on Oct. 12, 2022, in China National Intellectual Property Administration. The two applications are both incorporated by reference in their entirety.

### FIELD OF THE TECHNOLOGY

Embodiments of this application relate to the technical field of artificial intelligence and relate to, but not limited to, a method and apparatus for determining antigenic specificity, an electronic device, a storage medium and a computer program product.

### BACKGROUND OF THE DISCLOSURE

The human immune system includes congenital immunity and adaptive immunity. The adaptive immunity is an immune response which generates and targets initiation of a specific pathogen via contact with the specific pathogen (antigen). A T cell and a B cell are significant components for an adaptive immune system. Antigen recognition is one of the critical factors for T cell and B cell mediated immunity. The T cell and the B cell are mainly formed by interactions between a T cell receptor (TCR) and a B cell receptor (BCR) and the antigen. The TCR recognizes and binds with the antigen present on a major histocompatibility complex (MHC) on a cell membrane and the BCR directly binds with the specific antigen. Complementarity determining regions (CDR) on the TCR and BCR recognize and specifically bind with peptide molecules of the antigen. Sequencing the BCR or the TCR can be used for diagnosing malignant tumors of B lymphocytes or Tb lymphocytes and analyzing a post-treatment effect. Therefore, predicting the antigenic specificity of the TCR and the BCR has a transformative impact on many leading research fields such as treatment of infectious diseases and autoimmune diseases, and design of immune vaccines for cancers.

Often, predicting the antigenic specificity of the TCR and the BCR mainly includes methods based on manually defined features and artificial intelligence methods based on machine learning or deep learning. The methods based on manually defined features mainly classify or cluster a TCR sequence and a BCR sequence based on the manually defined features; and the methods based on artificial intelligence usually extract and learn the features automatically based on information such as the TCR and the BCR to predict the antigenic specificity of the TCR and the BCR.

However, the methods based on manually defined features can neither represent available information of the TCR sequence and the BCR sequence completely nor depict the distance difference between the sequences accurately. The methods based on artificial intelligence may obtain a better prediction result by training massive data with known antigen binding specificity as sample data. In a case that there is fewer sample data available, the prediction result is usually poor. Moreover, these methods cannot comprehensively characterize influence of TCR and BCR paired double strands on the TCR and the BCR during coding processing. Therefore, they cannot accurately predict the antigenic specificity of the TCR and the BCR.

### SUMMARY

Embodiments of this application provide a method and apparatus for determining antigenic specificity, an electronic device, a storage medium and a computer program product, which are at least applied to the artificial intelligence field and the medical field. The method and apparatus are capable of performing accurate word coding processing on double-stranded biological information of a cell receptor, and perform accurate feature extraction on the cell receptor based on an amino acid word sequence subject to word coding processing, to accurately determine the antigenic specificity of the cell receptor.

Technical solutions of the embodiments of this application are implemented as follows:

The embodiments of this application provide a method for determining antigenic specificity, executed by an electronic device. The method includes acquiring double-stranded biological information of a cell receptor; performing word coding processing on the double-stranded biological information to obtain an amino acid word sequence, the amino acid word sequence comprising an amino acid word representation; performing feature extraction on the cell receptor based on the amino acid word sequence with a pre-trained amino acid sequence prediction model to obtain an amino acid sequence representation of the cell receptor, the amino acid prediction model being trained with masked sample data; and determining the antigenic specificity of the cell receptor based on the amino acid sequence representations.

The embodiments of this application provide an electronic device, including: a memory, configured to store executable instructions; and a processor, configured to implement the method for determining antigenic specificity when executing the executable instructions stored in the memory.

Some embodiments of this application provide a non-transitory computer readable storage medium, storing the executable instructions, and configured to cause a processor to implement the method for determining antigenic specificity when executing the executable instructions.

Embodiments of this application have the following beneficial effects: word coding processing is performed on double-stranded biological information of a cell receptor to obtain an amino acid word sequence; feature extraction is performed on the cell receptor based on the amino acid word sequence with a pre-trained amino acid sequence prediction model to obtain an amino acid sequence representation of the cell receptor; the antigenic specificity of the cell receptor can be determined based on the amino acid sequence representations. Therefore, because the double-stranded biological information of the cell receptor is subject to the word coding processing, the influence of TCR and BCR paired double strands of the cell receptor on TCR and BCR during coding processing can be completely and comprehensively characterized, so that accurate word coding processing can be performed on gene information of the cell receptor. Moreover, because the amino acid sequence prediction model is obtained by training data obtained by mask processing of a portion of sample amino acid word representations in sample data, the amino acid sequence prediction model would not be converging too fast. Therefore, the accuracy of feature extraction on the cell receptor is further improved, which improves the accuracy of the antigenic specificity of the determined cell receptor.

### BRIEF DESCRIPTION OF THE DRAWINGS

FIG. 1 is an architectural schematic diagram of a system for determining antigenic specificity provided in an embodiment of this application.

FIG. 2 is a schematic structural diagram of an electronic device provided in an embodiment of this application.

FIG. 3 is an flow chart of a method for determining antigenic specificity provided in an embodiment of this application.

FIG. 4 is another flow chart of the method for determining antigenic specificity provided in some embodiments of this application.

FIG. 5 is a schematic diagram of an implementation flow chart of a method for training an amino acid sequence prediction model provided in an embodiment of this application.

FIG. 6 is a schematic diagram of an implementation flow chart of a data preprocessing method provided in an embodiment of this application.

FIG. 7 is a schematic diagram of a model framework of an algorithm corresponding to the method for determining antigenic specificity provided in some embodiments of this application.

FIG. 8 is a schematic diagram of a masking effect provided in an embodiment of this application before a mask strategy is modified.

FIG. 9 is a schematic diagram of a masking effect provided in an embodiment of this application after the mask strategy is modified.

FIG. 10 is a schematic diagram of performance comparison of an embodiment of this application on a public data set and other methods on TCR antigenic specificity recognition.

FIG. 11 is a schematic diagram of performance comparison of an embodiment of this application on the public data set and a single-stranded method on TCR antigenic specificity recognition.

FIG. 12 is a schematic diagram of performance comparison of an embodiment of this application on the public data set and other coding methods on TCR antigenic specificity recognition.

### DESCRIPTION OF EMBODIMENTS

To make the objectives, technical solutions, and advantages of this application clearer, the following describes this application in further detail with reference to the accompanying drawings. The described embodiments are not to be considered as a limitation to this application. All other embodiments obtained by a person of ordinary skill in the art without creative efforts shall fall within the protection scope of this application.

In the following description, the term “some embodiments” describes subsets of all possible embodiments, but it may be understood that “some embodiments” may be the same subset or different subsets of all the possible embodiments, and can be combined with each other without conflict. Unless otherwise defined, meanings of all technical and scientific terms used in the embodiments of this application are the same as those usually understood by those skilled in the art to which the embodiments of this application belong. The terms used in the embodiments of this application are merely intended to describe objectives of the specific embodiments, but are not intended to limit this application.

Prior to explaining the method for determining antigenic specificity in the embodiments of this application, some of the methods for determining antigenic specificity are described first.

Predicting the antigenic specificity of the TCR and the BCR mainly includes methods based on manually defined features and artificial intelligence methods based on machine learning or deep learning. The methods based on manual definition include: GLIPH (software for clustering TCR sequence functions), TCRdist (a tool kit which supports python API for analyzing an instruction set of TCR) and the like, which mainly classify or cluster TCR and BCR sequences based on the manually defined features. For example, TCRdist measures the distances among different cell receptors with a weighted Hamming distance for clustering the TCR. The methods based artificial intelligence include: DeepTCR (a python packet with a deep learning method with an unsupervised set and a supervised set for analyzing sequenced data of the T cell receptor), TCRAI, TCR-BERT (a model based on Transformer), which extract and learn features automatically based on information such as TCR and BCR sequences to predict the antigenic specificity of the TCR and the BCR. However, the methods based on manual definition, for example GLIPH unlearnability and TCRdist with characteristics such as the manually defined Hamming distance, have limitations and can neither represent available information of the TCR sequence and the BCR sequence completely nor depict the distance difference between the sequences accurately. The methods based on artificial intelligence, for example, DeepTCR and TCRAI, may obtain a better prediction result by training massive data with known antigen binding specificity. In a case that there is less data available, the prediction result is usually poor. Moreover, the TCR-BERT method pre-trains the TCR single-stranded data without comprehensively considering information of double strands in a pre-training stage. Therefore, coding cannot completely and comprehensively characterize the influence of TCR and BCR paired double strands on the TCR and the BCR. Moreover, it is merely the method for the TCR without considering the BCR.

The method for determining antigenic specificity in the embodiments of this application extracts and codes the sequence information of TCR/BCR based on a pre-trained language characterization model (Bidirectional Encoder Representation from Transformers (BERT)) of natural language processing for TCR/BCR antigenic specificity recognition. In the embodiments of this application, the double stranded TCR/BCR data is used for pre-training the BERT model for the first time to code a paired CDR3 sequence.

The method for determining antigenic specificity in the embodiments of this application includes the following steps: first, word coding processing is performed on double-stranded biological information of a cell receptor to obtain an amino acid word sequence, where the amino acid word sequence includes at least one amino acid word representation; Then, feature extraction is performed on the cell receptor based on the amino acid word sequence with a pre-trained amino acid sequence prediction model to obtain an amino acid sequence representation of the cell receptor, the amino acid prediction model is trained with masked sample data, and the masked sample data is data obtained by mask processing of a part of sample amino acid word representations in sample data; and finally, the antigenic specificity of the cell receptor is determined based on the amino acid sequence representations. Therefore, because the double-stranded biological information of the cell receptor is subjected to the word coding processing, the influence of cell receptor paired double strands on the double strands during coding processing can be completely and comprehensively characterized, so that accurate word coding processing can be performed on gene information of the cell receptor. Moreover, because the amino acid sequence prediction model is obtained by training data obtained by mask processing of a part of sample amino acid word representations in sample data, it can be ensured that the amino acid sequence prediction model will not be converged too fast. Therefore, the accuracy of feature extraction on the cell receptor is further improved, which guarantees the accuracy of the antigenic specificity of the determined cell receptor.

An electronic device in the embodiments of this application will be described below. The electronic device provided in the embodiments of this application may be implemented as either a terminal or a server. In an implementation, the electronic device provided in the embodiments of this application may be implemented as any terminal with a data processing function such as a notebook computer, tablet computer, a desktop computer, a mobile device (for example, a mobile phone, a portable music player, a personal digital assistant, a dedicated message device, a portable game device), an intelligent robot, an intelligent household electrical appliance and an intelligent vehicle-mounted device. In another implementation, the electronic device provided in the embodiments of this application may be implemented as a server. The server may be an independent physical server, a server cluster formed by a plurality of physical servers or a distributed system, and a cloud server which provides basic cloud computing services such as cloud servers, a cloud database, cloud computing, a cloud function, cloud storage, a network service, cloud communication, a middleware service, a domain name service, a safety service, a content delivery network (CDN), and a big data and artificial intelligence platform. The terminal and server may be directly or indirectly connected via a wired or wireless communication mode, which is not limited in the embodiments of this application. An electronic device implemented as the server will be described below.

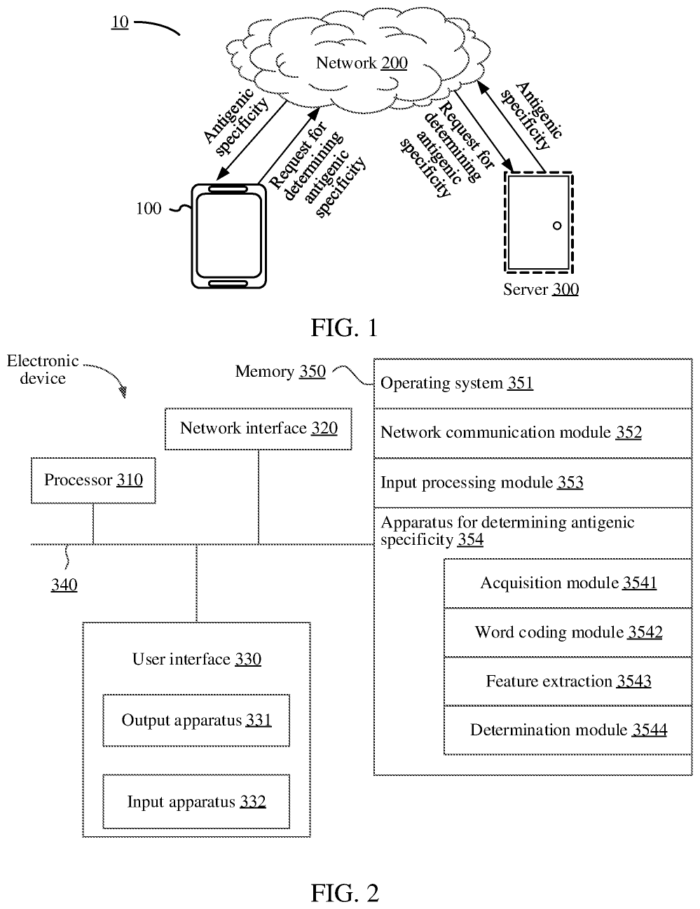

Referring to FIG. 1, FIG. 1 is an architectural schematic diagram of a system for determining antigenic specificity provided in an embodiment of this application. Some embodiments of this application is described by taking the method for determining antigenic specificity applied to an antigenic specificity prediction task of any one cell receptors of the TCR and the BCR as an example, that is to say, some embodiments of this application may be applied to the field of biomedicine or the treatment field of infectious diseases and autoimmune diseases, and the research and development and experimental stages in the field of design of immune vaccines for cancers to predict the antigenic specificity of the cell receptor. In some embodiments of this application, an antigenic specificity prediction platform of a cell receptor may be provided, and the antigenic specificity of the cell receptor may be predicted in the prediction platform. In an implementation process, the prediction platform may be implemented as an antigenic specificity prediction application. To support the antigenic specificity prediction application, the antigenic specificity prediction application is at least installed on the terminal in some embodiments of this application. The antigenic specificity prediction application is capable of using the method for determining antigenic specificity provided in some embodiments of this application to determine the antigenic specificity of the cell receptor.

In some embodiments of this application, the system 10 for determining antigenic specificity at least includes a terminal 100, a network 200 and a server 300, where the server 300 is a server for the antigenic specificity prediction application. The server 300 may form the electronic device in some embodiments of this application. The terminal 100 is connected to the server 300 through the network 200, and the network 200 may be either a wide area network or a local area network or a combination thereof. The terminal 100 acquires the double-stranded biological information of the cell receptor through a client of the antigenic specificity prediction application in response to that running the antigenic specificity prediction application, encapsulates the double-stranded biological information of the cell receptor into a request for determining antigenic specificity, and transmits the request for determining antigenic specificity to the server 300. The server 300 analyzes the request for determining antigenic specificity to obtain the double-stranded biological information, and performs word coding processing on the double-stranded biological information of the cell receptor to obtain the amino acid word sequence, where the amino acid word sequence includes at least one amino acid word representation; Then, feature extraction is performed on the cell receptor based on the amino acid word sequence with a pre-trained amino acid sequence prediction model to obtain an amino acid sequence representation of the cell receptor, where the amino acid sequence prediction model is obtained by training data obtained by mask processing of a part of sample amino acid word representations in sample data, i.e., the amino acid sequence prediction model is trained with the masked sample data, where the masked sample data is data obtained by mask processing of a part of sample amino acid word representations in sample data; and finally, the antigenic specificity of the cell receptor is determined based on the amino acid sequence representations. Upon obtaining the antigenic specificity of the cell receptor, the server 300 transmits the antigenic specificity of the cell receptor to the terminal 100 for reference studies by researchers.

In some embodiments, the method for determining antigenic specificity may further be implemented with the terminal 100. The method may include the following steps: the terminal as an executive body acquires the double-stranded biological information of the cell receptor; word coding processing is performed on double-stranded biological information of a cell receptor to obtain an amino acid word sequence; then the terminal predicts the amino acid sequence of the cell receptor based on the amino acid word sequence to obtain amino acid sequence representations of the cell receptor; and finally, the terminal determines the antigenic specificity of the cell receptor based on the amino acid sequence representations.

The method for determining antigenic specificity provided in some embodiments of this application may further be implemented based on the cloud platform and through the cloud technology. For example, the server 300 may be a cloud server. The cloud server performs word coding processing on the double-stranded biological information of the cell receptor, or the cloud server predicts the amino acid sequence of the cell receptor based on the amino acid word sequence, or the cloud server determines the antigenic specificity of the cell receptor based on the amino acid sequence representations.

In some embodiments, there may further be a cloud memory where the double-stranded biological information of the cell receptor may be stored, or the amino acid word sequence may further be stored in the cloud memory, or the predicted amino acid sequence representations may further be stored in the cloud memory. Accordingly, in response to that the antigenic specificity of the cell receptor is determined, the amino acid sequence representations may be acquired from the cloud memory, so that the antigenic specificity of the cell receptor is directly determined.

Cloud technology is a hosting technology which unifies series of resources such as hardware, software and networks in the wide area network or the local area network to implement data computing, storing, processing and sharing. The cloud technology is a generic term of a network technology, an information technology, an integrated technology, a management platform technology and an application technology based on a cloud computing business model, and may form a resource pool which is used on demand and is flexible and convenient. Cloud computing technology will become a vital support. Background services of a technical network system need a lot of computing and storing resources such as video websites, photo websites and more portal websites. With high development and application of internet industry, each article may have its own recognition mark in the future and the recognition mark needs to be transmitted to a background system for logic processing. Data in different degree levels will be separately processed. Various industrial data all need powerful system backing, which may only be implemented by cloud computing.

FIG. 2 is a schematic structural diagram of an electronic device provided in some embodiments of this application. The electronic device shown in FIG. 2 may be a device for determining antigenic specificity. The electronic device includes at least one processor 310, a memory 350, at least one network interface 320 and a user interface 330. Components in the electronic device are coupled together through a bus system 340. It may be understood that the bus system 340 is configured to implement connection and communication between the components. In addition to a data bus, the bus system 340 further includes a power bus, a control bus, and a state signal bus. But, for case of clear description, all types of buses in FIG. 2 are marked as the bus system 340.

The processor 310 may be an integrated circuit chip with a signal processing capacity such as a universal processor, a digital signal processor (DSP) or another programmable logic device, a discrete gate or transistor logic device, and a discrete hardware component, where the universal processor may be a microprocessor or any conventional processor.

The user interface 330 includes one or more output apparatuses 331 capable of presenting a medium content, and one or more input apparatuses 332.

Memory 350 may be a removable memory, an irremovable memory or a combination thereof. A hardware device includes a solid state memory, a hard disk drive and an optical disk drive. The memory 350 may include one or more storage devices away from the processor 310 in physical location. Memory 350 includes a volatile memory or a non-volatile memory, or may include both a volatile memory and a non-volatile memory. The non-volatile memory may be a read-only memory (ROM), and the volatile memory may be a random access memory (RAM). The memory 350 described in some embodiments of this application is intended to include any suitable type of memory. In some embodiments, memory 350 is capable of storing data to support various operations. Examples of these data include a program, a module and a data structure or a subset or a superset thereof, which will be exemplarily described below.

An operating system 351 includes a system program, configured to process various basic system services and execute hardware related tasks, for example, a framework layer, a core library layer and a driving layer for implementing various basic businesses and processing tasks based on hardware;

a network communication module 352 is configured to arrive at other computing devices via one or more (wired or wireless) network interfaces 320. Network interface 320 may include: Bluetooth, wireless fidelity (WiFi) and a universal serial bus (USB);

An input processing module 353 is configured to detect one or more user inputs or interactions from one or more input apparatuses 332 and to translate the detected inputs or interactions.

In some embodiments, the apparatus provided in may be implemented by software. FIG. 2 shows apparatus 354 for determining antigenic specificity stored in the memory 350. The apparatus 354 for determining antigenic specificity may be an apparatus for determining antigenic specificity in the electronic device, which may be software in the form of a program and plug-in, including the following software modules: an acquisition module 3541, a word coding module 3542, a feature extraction module 3543 and a determination module 3544, where these modules are logic, so that they may be arbitrarily combined or may be further split according to implemented functions. Functions of each module will be described below.

In some other embodiments, the apparatus provided may be implemented by software. As an example, the apparatus provided in some embodiments of this application may be a processor in the form of a hardware coding processor, which is programmed to execute the method for determining antigenic specificity provided in some embodiments of this application. For example, the processor in the form of the hardware coding processor may be one or more application specific integrated circuits (ASIC), a DSP, a programmable logic device (PLD), a complex programmable logic device (CPLD), a field programmable gate array (FPGA) or another electronic element.

The method for determining antigenic specificity provided in each embodiment of this application may be executed by the electronic device, where the electronic device may be either any one terminal with the data processing function or the server, i.e., the method for determining antigenic specificity provided in each embodiment of this application may be executed by either the terminal or the server, or the method may further be executed by interaction of the terminal and the server.

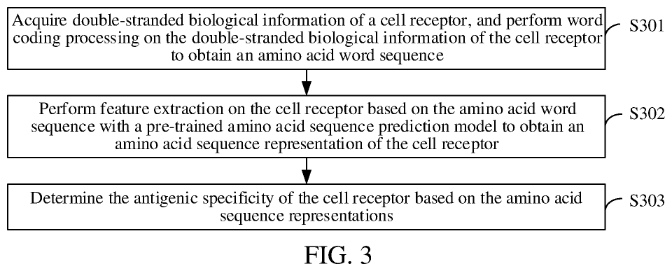

Referring to FIG. 3, FIG. 3 is a flow chart of the method for determining antigenic specificity provided in an embodiment of this application. The method will be described in combination with steps shown in FIG. 3. The method for determining antigenic specificity in FIG. 3 is described by taking the server as the executive body, including the following steps S301 to S303.

Step S301: Double-stranded biological information of a cell receptor is acquired, and word coding processing is performed on double-stranded biological information of the cell receptor to obtain an amino acid word sequence.

Here, the amino acid word sequence includes at least one amino acid word representation. The cell receptor may be an adaptive immune receptor and includes a T cell receptor (TCR) or a B cell receptor (BCR). The TCR is a characteristic mark on the surface of all T cells and binds with CD3 via a noncovalent bond to form a TCR-CD3 complex; the TCR plays a role of recognizing the antigen. The TCR is a heterodimer formed by two different peptide chains: an α chain and a β chain. Each peptide chain may further be divided into a variable region (V region), a constant region (C region), a transmembrane region and a cytoplasmic region. TCR molecules belong to an immune globulin superfamily, and the antigenic specificity of the TCR exists in the V region; the V region has three hypervariable regions CDR1, CDR2 and CDR3, where variation in CDR3 is the maximum, which directly decides the antigen binding specificity of the TCR. In a case that the TCR recognizes an MHC-antigen peptide complex, the CDR1 and CDR2 recognize and bind with a side wall of an MHC molecular antigen binding cleft and the CDR3 directly binds with the antigen peptide.

The TCR is divided into two categories: TCR1 and TCR2; the TCR1 includes two chains: a γ chain and α chain, and the TCR2 includes two chains: an α chain and a β chain. In some embodiments of this application, in a case that the cell receptor is the TCR1, the double-stranded biological information is amino acid information carried on the γ chain and the δ chain; and in a case that the cell receptor is the TCR2, the double-stranded biological information is amino acid information carried on the α chain and the β chain. In an implementation, the double-stranded biological information may be amino acid sequence information of the CDR3 region of the TCR.

BCR is a molecule located on the surface of the B cell, responsible for specifically recognizing and binding with the antigen. The BCR is essentially a surface membrane immunoglobulin (mIg). The BCR has the antigen binding specificity (i.e., the antigenic specificity). The BCR diversity of each individual reaches up to 5×10¹³. A BCR library with a huge capacity is formed. The individual is endowed with a huge potential to recognize various antigens and generate specific antibodies. Two types of surface membrane immunoglobulins will be expressed on the surface of the B cell: mIgM and mIgD, where the mIgM and mIgD are used for specifically recognizing and binding with the antigen. mIg is formed by connecting two heavy chains and two light chains. Each of the heavy chains is divided into a variable region (V region, with about 110 amino acid residues), a constant region (C region, with about 330 amino acid residues), a transmembrane region (26 amino acid residues) and a cytoplasmic region (3 amino acid residues); each of the light chains only has the V region and the C region. The V regions of the heavy chain and the light chain each have three regions with highly variable amino acid compositions and arrangement orders. These regions are capable of forming a complementary spatial conformation with the antigen epitope, which is called the complementarity determining region (CDR). The three CDRs on mIg are all involved in recognition of the antigen and jointly decide the antigenic specificity of the BCR. In some embodiments of this application, in a case that the cell receptor is the BCR, the double-stranded biological information is amino acid information carried on the heavy chain and the light chain. In an implementation, the double-stranded biological information may be amino acid sequence information of the CDR3 region of the BCR.

The word coding processing is a process of coding the double-stranded biological information into a sentence. Because words in the biological sequence have no clear conceptions, a fixed quantity of amino acids may be selected from the double-stranded biological information as a word to form a sentence.

In some embodiments of this application, in a case that the fixed quantity of amino acids is coded into a word, the fixed quantity may be determined based on the preset monomeric unit quantity. For example, k monomeric units (kmer) may be selected as the monomeric unit quantity to code the amino acid sequence information in the double-stranded biological information in sequence.

In some embodiments, in a case that the word coding processing is performed, there is a certain quantity of overlapping amino acids between the two adjacent amino acid word representations. Here, the quantity of the overlapping amino acids may be a value obtained by subtracting 1 from the used monomeric unit quantity during coding. In a case that the amino acid sequence information includes N amino acids, N−2 amino acid word representations may be obtained through the word coding processing, so that the N−2 amino acid word representations form an amino acid word sequence, i.e., the above sentence. The sentence may be inputted into the amino acid sequence prediction model as an input text in a subsequent process of determining the antigenic specificity.

Step S302: Feature extraction is performed on the cell receptor based on the amino acid word sequence with a pre-trained amino acid sequence prediction model to obtain an amino acid sequence representation of the cell receptor.

In some embodiments of this application, the feature extraction on the cell receptor may be to predict the amino acid sequence to determine the amino acid sequence representations corresponding to the double-stranded biological information. The amino acid sequence of the cell receptor may be predicted with the amino acid sequence prediction model. In an implementation process, the amino acid word sequence may be inputted into the amino acid sequence prediction model as the input data of the amino acid sequence prediction model, and the amino acid sequence representations of the cell receptor are outputted by the amino acid sequence prediction model. The amino acid sequence prediction model is obtained by training data obtained by mask processing of a portion of sample amino acid word representations in sample data. The amino acid sequence prediction model is trained with the masked sample data, where the masked sample data is data obtained by mask processing of a part of sample amino acid word representations in sample data.

Here, because the amino acid sequence prediction model is the pre-trained prediction model, in a case that the amino acid sequence representations are predicted, the amino acid sequence representations of the cell receptor may be accurately predicted. Moreover, because in a case that the amino acid sequence prediction model is trained, a part of sample amino acid word representations will be masked for the input sample data, the amino acid sequence prediction model predicts the masked part, so that a self-learning process of the masked part is performed based on the input sample data. Therefore, it may be ensured that the model will not be converged too fast, and parameters in the model may also be accurately learned.

S303: the antigenic specificity of the cell receptor is determined based on the amino acid sequence representations.

In some embodiments of this application, upon obtaining the amino acid sequence representations of the cell receptor, the antigenic specificity of the cell receptor is determined based on the amino acid sequence representations of the cell receptor.

Here, the antigenic specificity is the most prominent characteristic of the immune response, which is the theoretical foundation for immunologic diagnosis and prevention and treatment. The antigenic specificity is the property that the antigen may only bind with corresponding antibodies and sensitized lymphocytes. The antigenic specificity is expressed in either immunogenicity or immunoreactivity. The antigenic specificity expressed in immunogenicity means that a certain specific antigen only causes a certain specific immune response. The antigenic specificity expressed in immunoreactivity means that a certain specific antigen only binds with at least one of the corresponding antibodies and specific sensitized lymphocytes to show response. The antigenic specificity first depends on its chemical composition. However, in spite of the same chemical composition, its specificity is also different due to a difference in spatial configuration.

In some embodiments of this application, upon obtaining the amino acid sequence of the cell receptor, i.e., the chemical composition and the spatial configuration of the cell receptor, the cell receptor may be subjected to classifying processing based on the chemical composition and the spatial configuration of the cell receptor, so that the antigenic specificity of the cell receptor is mapped.

In an implementation process, mapping processing (i.e., classification processing) may be performed on characteristics corresponding to the amino acid sequence through a classification head including a multilayer perceptron (MLP) to obtain the antigenic specificity of the cell receptor. In some embodiments of this application, the MLP may be a full connected network.

The method for determining antigenic specificity in the embodiments of this application includes the following steps: word coding processing is performed on double-stranded biological information of a cell receptor to obtain an amino acid word sequence, feature extraction is performed on the cell receptor based on the amino acid word sequence with a pre-trained amino acid sequence prediction model to obtain an amino acid sequence representation of the cell receptor; The amino acid sequence prediction model is obtained by training data obtained by mask processing of a part of sample amino acid word representations in sample data, i.e., the amino acid sequence prediction model is trained with the masked sample data, where the sample data is data obtained by mask processing of a part of sample amino acid word representations in sample data; and the antigenic specificity of the cell receptor can be determined based on the amino acid sequence representations. In some embodiments of this application, because the double-stranded biological information of the cell receptor is subjected to the word coding processing, the influence of cell receptor paired double strands on the double strands during coding processing can be completely and comprehensively characterized, so that accurate word coding processing can be performed on gene information of the cell receptor. Moreover, because the amino acid sequence prediction model is obtained by training data obtained by mask processing of a part of sample amino acid word representations in sample data, it can be ensured that the amino acid sequence prediction model will not be converged too fast during predicting of the amino acid sequence. Therefore, the accuracy of predicting of the amino acid sequence on the cell receptor is further improved, which guarantees the accuracy of the antigenic specificity of the determined cell receptor.

In some embodiments of this application, the method for determining antigenic specificity may be applied to an antigenic specificity prediction task of any one cell receptors of TCR and BCR, that is to say, some embodiments of this application may be applied to the field of biomedicine or the treatment field of infectious diseases and autoimmune diseases, and the research and development and experimental stages in the field of design of immune vaccines for cancers, to predict the antigenic specificity of the cell receptor. In some embodiments of this application, an antigenic specificity prediction platform of a cell receptor may be provided, and the antigenic specificity of the cell receptor may be predicted in the prediction platform. In an implementation process, the prediction platform may be implemented as an antigenic specificity prediction application. The system for determining antigenic specificity at least includes the terminal and the server. The antigenic specificity prediction application is at least installed on the terminal in some embodiments of this application. The antigenic specificity prediction application is capable of determining the antigenic specificity of the cell receptor based on the method for determining antigenic specificity provided in some embodiments of this application. The server may be a sever of the antigenic specificity prediction application.

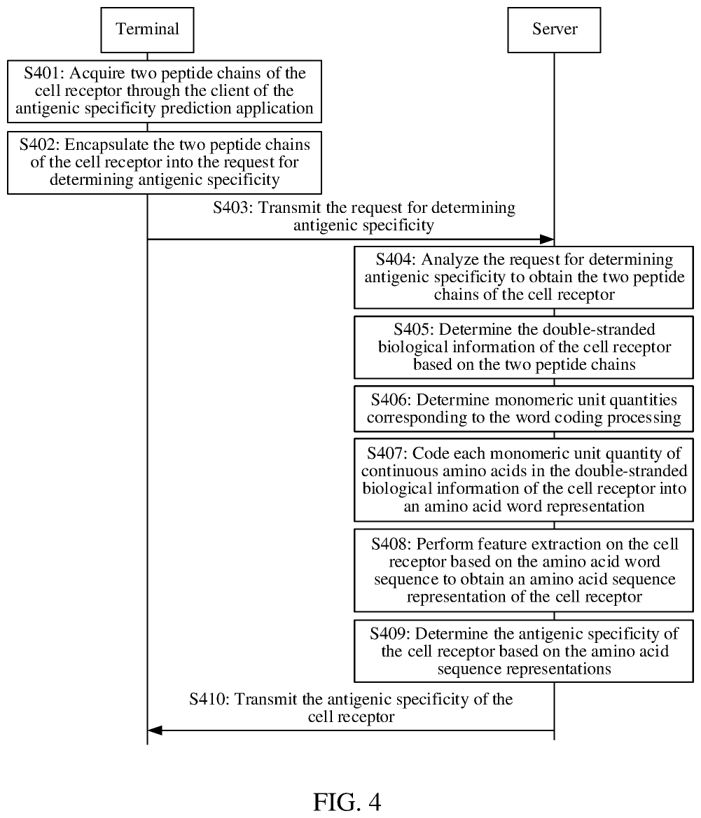

FIG. 4 is another flow chart of the method for determining antigenic specificity provided in some embodiments of this application. As shown in FIG. 4, the method includes the following steps S401 to S410:

Step S401: The terminal acquires two peptide chains of the cell receptor through the client of the antigenic specificity prediction application.

In some embodiments of this application, the two peptide chains of the cell receptor may be acquired through an experiment, and corresponding peptide information corresponding to the two peptide chains are obtained correspondingly.

Step S402: The terminal encapsulates the two peptide chains of the cell receptor into the request for determining antigenic specificity.

Here, the peptide chain information corresponding to the two peptide chains may be encapsulated into the request for determining antigenic specificity.

Step S403: The terminal transmits the request for determining antigenic specificity to the server.

Step S404: The server analyzes the request for determining antigenic specificity to obtain the two peptide chains of the cell receptor.

In some embodiments, the cell receptor includes a T cell receptor or a B cell receptor; In a case that the cell receptor is the T cell receptor, the two peptide chains includes an α chain and a β chain; in a case that the cell receptor is the B cell receptor, the two peptide chains include a heavy chain and a light chain.

Step S405: The server determines the double-stranded biological information of the cell receptor based on the two peptide chains.

In some embodiments of this application, the two peptide chains may be sequenced to obtain the double-stranded biological information of the cell receptor.

Step S406: The server determines monomeric unit quantities corresponding to the word coding processing.

Here, each of the monomeric unit quantities is the quantity of the amino acids included in each amino acid word representation during word coding processing.

Step S407: The server codes each monomeric unit quantity of continuous amino acids in the double-stranded biological information of the cell receptor into an amino acid word representation.

Here, the double-stranded biological information is coded to obtain a plurality of amino acid word representations, and the obtained plurality of amino acid word representations form the amino acid word sequence. In the amino acid word sequence, there is a preset quantity of overlapping amino acids between two adjacent amino acid word representations, and the preset quantity is 1 less than the monomeric unit quantity. The amino acid word sequence includes at least one amino acid word representation.

In some embodiments, the value of the monomeric unit quantities may be 3. Therefore, there is two overlapping amino acids between the two adjacent amino acid word representations.

In some embodiments, the double-stranded biological information includes gene information corresponding to each of the peptide chains in the two peptide chains. In step S407, the step of coding each monomeric unit quantity of continuous amino acids in the double-stranded biological information of the cell receptor into an amino acid word representation may be implemented in the following ways: first, each monomeric unit quantity of continuous amino acids in each of the peptide chains is coded into an amino acid word representation to correspondingly form an amino acid subsequence corresponding to each of the peptide chains; and then, the amino acid word sequence is determined according to the amino acid subsequences corresponding to the two peptide chains.

Here, the amino acid subsequences corresponding to the two peptide chains may be spliced to obtain a spliced word sequence; then, the spliced word sequence is successively subjected to marking processing, segmenting processing and location coding processing to obtain the amino acid word sequence. That is to say, the spliced word sequence may be subjected to marking processing to obtain a marked spliced word sequence; then, the marked spliced word sequence is subjected to segmenting processing to obtain a segmented spliced word sequence; and finally, location coding processing is performed on the segmented spliced word sequence to obtain the amino acid word sequence.

In some embodiments of this application, the splicing processing refers to connecting two amino acid subsequences corresponding to the two peptide chains to form an amino acid word sequence, where the sequence length of the amino acid word sequence is equal to the sum of the sequence lengths of the two amino acid subsequences. In an implementation process, in a case that the cell receptor is the T cell receptor, the two peptide chains include the α chain and the β chain. Therefore, the amino acid subsequences corresponding to the two peptide chains are respectively the amino acid subsequence corresponding to the α chain and the amino acid subsequence corresponding to the β chain. During splicing processing, the amino acid subsequence corresponding to the β chain may be spliced to the amino acid subsequence corresponding to the a chain to form an amino acid word sequence. In an implementation process, in a case that the cell receptor is the B cell receptor, the two peptide chains include the light chain and the heavy chain. Therefore, the amino acid subsequences corresponding to the two peptide chains are respectively the amino acid subsequence corresponding to the light chain and the amino acid subsequence corresponding to the heavy chain. During splicing processing, the amino acid subsequence corresponding to the heavy chain may be spliced to the amino acid subsequence corresponding to the light chain to form an amino acid word sequence.

The marking processing refers to marking at least one amino acid word with specific functions and meaning in the amino acid word sequence to obtain a marked label, to determine the amino acid word representation based on the marked label in subsequent predicting and processing processes. The segmenting processing refers to dividing the amino acid word sequence as a sentence component into a plurality of amino acid word sequence segments, to perform prediction based on the plurality of amino acid word sequence segments in a subsequent amino acid sequence prediction process. Location coding processing refers to performing coding processing on the locations of the amino acid word representations in the amino acid word sequence to obtain location coding information of each of the amino acid word representations of the amino acid word sequence. For example, in a case that the third amino acid word representation in the amino acid word sequence is subjected to the location coding processing, a location “3” of the amino acid word representation may be coded to the location coding information of the amino acid word representation, so that the location information of the corresponding amino acid word representation in the amino acid word sequence may be acquired by performing coding processing on the location coding information.

Step S408: The server performs feature extraction on the cell receptor based on the amino acid word sequence to obtain an amino acid sequence representation of the cell receptor.

Step S409: The server determines the antigenic specificity of the cell receptor is determined based on the amino acid sequence representations.

S410: the server transmits the antigenic specificity of the cell receptor to the terminal.

The method for determining antigenic specificity provided in some embodiments of this application performs word coding processing on the double-stranded biological information of the cell receptor based on the monomeric unit quantities, where each monomeric unit quantity of continuous amino acids is coded into an amino acid word representation to form the amino acid word sequence, and there is a preset quantity of overlapping amino acids between two adjacent amino acid word representations, and the preset quantity is 1 less than the monomeric unit quantity. Thus, to code the continuous and overlapping amino acids in the double-stranded biological information is capable of guaranteeing accurate coding of each amino acid in the double-stranded biological information and prevent omission of information of any one amino acid in the double-stranded biological information, so that the accuracy of the word coding processing is guaranteed.

In some embodiments, feature extraction may further be performed on the cell receptor based on the amino acid word sequence with the pre-trained amino acid sequence prediction model to obtain the amino acid sequence representation of the cell receptor. The amino acid sequence prediction model includes a word coding processing layer, a mask processing layer and a prediction processing layer.

In a using process of the amino acid sequence prediction model, the word coding processing layer is configured to implement the step of performing the word coding processing on the double-stranded biological information of the cell receptor in the above step S301 or to implement the step of performing the word coding processing in steps S405 to S407 to implement the word coding processing on the double-stranded biological information of the cell receptor, to obtain the amino acid word sequence. The mask processing layer is configured to implement the step of performing mask processing on the at least one sample amino acid word representation in the sample amino acid word sequence to obtain a masked sample amino acid word sequence. The prediction processing layer is configured to implement the step of predicting the amino acid sequence of the cell receptor based on the amino acid word sequence in the above step S303 to obtain the amino acid sequence representations of the cell receptor.

In a training process of the amino acid sequence prediction model, the word coding processing layer is configured to implement the step of performing word coding processing on the sample double-stranded biological information of the sample receptor to obtain a sample amino acid word sequence. The mask processing layer is configured to implement mask processing on at least one sample amino acid word representation in the sample amino acid word sequence to obtain a masked sample amino acid word sequence. The prediction processing layer is configured to implement amino acid sequence prediction on a sample cell receptor based on the masked sample amino acid word sequence to obtain a sample amino acid sequence representation of the sample cell receptor.

The training process of the amino acid sequence prediction model will be described below. A method for training the amino acid sequence prediction model provided in some embodiments of this application may be executed with a model training module. The model training module may be a module in the device (i.e., the electronic device) for determining antigenic specificity, i.e., the model training module may be either the server or the terminal; or, the model training module may also be another device independent of the device for determining antigenic specificity, i.e., the model training module is another electronic device distinguished from the server and the terminal configured to implement the method for determining antigenic specificity.

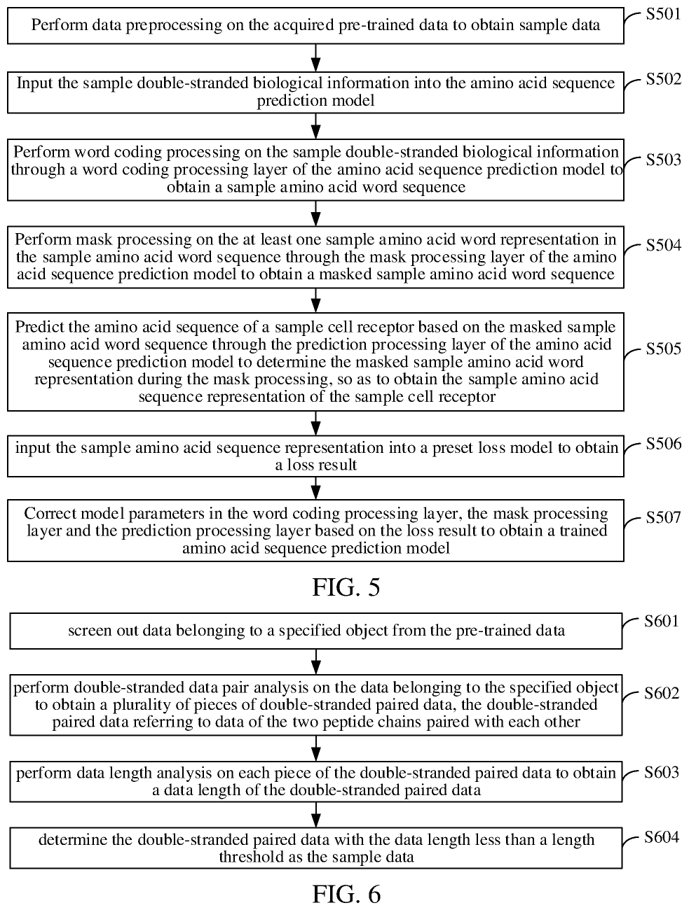

FIG. 5 is a schematic diagram of an implementation flow chart of the method for training the amino acid sequence prediction model provided in some embodiments of this application. As shown in FIG. 5, the amino acid sequence prediction model may be trained by cyclically iterating the following steps S501 to S507 till the amino acid sequence prediction model satisfies a preset condition of convergence to be converged. As shown in FIG. 5, the method includes the following steps S501 to S507:

Step S501: Data preprocessing is performed on the acquired pre-trained data to obtain sample data.

Here, the sample data includes sample double-stranded biological information of the sample cell receptor.

Step S502: The sample double-stranded biological information is inputted into the amino acid sequence prediction model.

Step S503: Word coding processing is performed on the sample double-stranded biological information through the word coding processing layer of the amino acid sequence prediction model to obtain a sample amino acid word sequence, where the sample amino acid word sequence includes at least one sample amino acid word representation.

Step S504: Mask processing is performed on the at least one sample amino acid word representation in the sample amino acid word sequence through the mask processing layer of the amino acid sequence prediction model to obtain a masked sample amino acid word sequence.

Here, the mask processing refers to mask the part of sample amino acid word representations in the sample amino acid word sequence, that is to say, prior to predicting the sample amino acid sequence representations, information of the sample amino acid word representations at partial locations in front of and behind the current sample amino acid word representation is masked for the part of sample amino acid word representations in the sample amino acid word sequence.

In some embodiments of this application, the amino acid sequence of the sample cell receptor may be predicted based on the masked amino acid word sequence with the amino acid sequence prediction model to obtain the sample amino acid sequence representations of the sample cell receptor. The amino acid sequence prediction model may be an amino acid sequence prediction model implemented based on a BERT technology.

We describe below why it is needed to mask the part of sample amino acid word representations in some embodiments of this application.

BERT needs masking because it uses a Transformer module. Therefore, to know why BERT needs masking is to determine why the Transformer module needs masking. However, the two are different: because BERT only uses the coding part in the Transformer module rather than decoding part. Therefore, compared with two kinds of mask processing (key padding mask and attention mask) in the Transformer module, BERT only has the key padding mask, i.e., it ignores information in the padding part. In the decoding stage of the Transformer module, it is also needed to ignore information behind the current location, so that the attention mask is also needed. In short, the amino acid sequence prediction model performs mask processing on the part of words in a case that the sentence is coded during pre-training, which plays a major role of guessing what word is masked with the words in front of and behind the masked word. Because the word is manually masked, a computer knows the correct value of the masked word, so that whether the word guessed by the amino acid sequence prediction model is accurate may also be judged.

In some embodiments of this application, in the mask processing process, the sample amino acid word representations at a preset masking proportion (for example, 15%) may be masked, and then the amino acid sequence prediction model predicts the masked sample amino acid word representations. The amino acid sequence prediction model will process each sample amino acid word representation individually, i.e., not consider word group information during mask processing. For example, for the sentence “the author of Jing Ye Si is Li Bai”, the BERT module may perform mask processing to obtain “the author of Jing Ye Si is [Mask] Bai”. For the amino acid sequence prediction model in some embodiments of this application, assuming that the sample amino acid word sequence is “CAVPGNNDMRF”, the amino acid sequence prediction model may perform mask processing to obtain “[Mask][Mask][Mask]PGN GNN NND NDM DMR MRF”.

In some embodiments of this application, by masking some word groups (i.e., the sample amino acid word representations) in the sentence formed by the sample amino acid word sequence and predicting the whole word groups, the amino acid sequence prediction model may better capture relationships between the word groups and substances.

In some embodiments, mask processing may be performed on the part of sample amino acid word representations in the sample amino acid word sequence in the following ways: first, at least one sample amino acid word representation randomly selected is determined as a target amino acid word representation from the sample amino acid word sequence; then adjacent amino acid word representations adjacent to the target amino acid word representation are determined; and finally, mask processing is performed on overlapping amino acids in the target amino acid word representation and the first adjacent amino acid word representation and overlapping amino acids in the second adjacent amino acid word representation to obtain the masked amino acid word sequence.

In some embodiments of this application, the mask processing may be implemented in a random masking method, that is to say, a certain quantity of sample amino acid word representations are randomly selected for the mask processing. In some embodiments, the masking proportion may be pre-determined or preset. The masking proportion refers to a ratio between the quantity of the determined target amino acid word representations and the total quantity of all sample amino acid word representations in the sample amino acid word sequence. Then, the target quantity of the target amino acid word representations is determined based on the masking proportion and the total quantity, and the target quantity of the target amino acid word representations are randomly determined from all the sample amino acid word representations based on data of the determined target amino acid word representations. For example, the masking proportion may be set to be 15%. Therefore, during the mask processing, 15% of the target amino acid word representations may be masked.

The adjacent amino acid word representations include: a first adjacent amino acid word representation adjacent to a first side of the target amino acid word representation and a second adjacent amino acid word representation adjacent to a second side of the target amino acid word representation, where the first side and the second side refer to two opposite sides of the target amino acid word representation. For example, the first side may be a side where the amino acid word representation in front of the target amino acid word representation is located, and the second side may be a side where the amino acid word representation behind the target amino acid word representation is located.

In some embodiments, in a case that the target amino acid word representation is located at a sequence starting location of the sample amino acid word sequence, the adjacent amino acid word representations include a second adjacent amino acid word representation adjacent to the second side of the target amino acid word representation, and in this case, there is no first adjacent amino acid word representation adjacent to the first side of the target amino acid word representation; and in a case that the target amino acid word representation is located at a sequence end location of the sample amino acid word sequence, the adjacent amino acid word representations include a first adjacent amino acid word representation adjacent to the first side of the target amino acid word representation, and in this case, there is no second adjacent amino acid word representation adjacent to the second side of the target amino acid word representation.

The overlapping amino acids refer to names of the amino acid representations corresponding to the overlapping amino acids in the two adjacent sample amino acid word representations. Because each sample amino acid word representation is formed by coding the monomeric unit quantity of continuous amino acids, there is a preset quantity of overlapping amino acids between two adjacent sample amino acid word representations, and the preset quantity is 1 less than the monomeric unit quantity. For the target amino acid word representation and the first adjacent amino acid word representation, the overlapping amino acids refer to the preset quantity of amino acids, close to the target amino acid word representation, in the first adjacent amino acid word representation or the preset quantity of amino acids, close to the first adjacent amino acid word representation, in the target amino acid word representation; that is, the preset quantity of amino acids in the back in the first adjacent amino acid word representation or the preset quantity of amino acids in the front in the target amino acid word representation. For the target amino acid word representation and the second adjacent amino acid word representation, the overlapping amino acids refer to the preset quantity of amino acids, close to the target amino acid word representation, in the second adjacent amino acid word representation or the preset quantity of amino acids, close to the second adjacent amino acid word representation, in the target amino acid word representation; that is, the preset quantity of amino acids in the front in the second adjacent amino acid word representation or the preset quantity of amino acids in the back in the target amino acid word representation.

For example, in a case that the monomeric unit quantity is 3, the preset quantity is 2. For the target amino acid word representation and the first adjacent amino acid word representation, the overlapping amino acids refer to the last two amino acids in the first adjacent amino acid word representation or the first two amino acids in the target amino acid word representation; and for the target amino acid word representation and the second adjacent amino acid word representation, the overlapping amino acids refer to the first two amino acids in the second adjacent amino acid word representation or the last two amino acids in the target amino acid word representation.

In some embodiments of this application, because the mask processing is performed on the part of sample amino acid word representations after the word coding processing, it may be guaranteed that the amino acid sequence prediction model will not be converged too fast during amino acid sequence prediction, so that the accuracy for predicting the amino acid sequence of the cell receptor is further improved.

Step S505: The amino acid sequence of the sample cell receptor is predicted based on the masked sample amino acid word sequence through the prediction processing layer of the amino acid sequence prediction model to determine the masked sample amino acid word representation during the mask processing, to obtain the sample amino acid sequence representation of the sample cell receptor.

In some embodiments, upon obtaining the sample amino acid sequence representation, the sample antigenic specificity of the sample cell receptor is determined based on the sample amino acid sequence representation. Due to the multimodal feature of the sample amino acid sequence representation, determining the sample antigenic specificity of the sample cell receptor may be implemented by the multilayer perceptron.

Here, the sample amino acid sequence representation may be inputted into the multilayer perceptron; and then, mapping processing is performed on the multimodal feature corresponding to the sample amino acid sequence representation through the multilayer perceptron to obtain the sample antigenic specificity of the sample cell receptor.

In an implementation, the multilayer perceptron may be a fully connected network which may be connected behind the amino acid sequence prediction model. Upon predicting the sample amino acid sequence representation of the sample cell receptor by the amino acid sequence prediction model, the sample amino acid sequence representation is a sequence representation including a feature with multiple models, and in this case, the sample amino acid sequence representation may be inputted into the multilayer perceptron as an input feature of the multilayer perceptron. Mapping processing is performed on the multimodal feature corresponding to the sample amino acid sequence through the multilayer perceptron to obtain the sample antigenic specificity of the sample cell receptor. The mapping processing may be any type of classification processing, and the antigenic affinity of the sample cell receptor may be classified based on the multimodal feature corresponding to the sample amino acid sequence to obtain an antigenic affinity result corresponding to the sample cell receptor, to determine the sample antigenic specificity of the sample cell receptor based on the antigenic affinity result.

Step S506: The sample amino acid sequence representation is inputted into a preset loss model to obtain a loss result.

In some embodiments, step S506 may be implemented in the following ways: first, the sample amino acid sequence representation and the sample double-stranded biological information are inputted into the preset loss model; then, a sequence distance between the sample amino acid sequence representation and the sample double-stranded biological information is determined through a cross entropy loss function in the preset loss model; and finally, the loss result is determined according to the sequence distance.

In some embodiments of this application, the preset loss model includes the cross entropy loss function, through which the amino acid prediction model is trained in an end-to-end manner. The predicted sequence distance between the sample amino acid sequence representation and the sample double-stranded biological information (i.e., the true amino acid sequence representation) may be calculated by the cross entropy loss function. Here, the greater the sequence distance is, correspondingly, the smaller the similarity between the sample amino acid sequence representation and the sample double-stranded biological information is; and the smaller the sequence distance is, correspondingly, the greater the similarity between the sample amino acid sequence representation and the sample double-stranded biological information is.

In some embodiments of this application, the sequence distance between the sample amino acid sequence representation and the sample double-stranded biological information is calculated by the loss function in the preset loss model to obtain a loss result, so that the difference between the predicted result and the true result of the amino acid sequence prediction model with the current model parameters may be determined accurately.

Step S507: Model parameters in the word coding processing layer, the mask processing layer and the prediction processing layer are corrected based on the loss result to obtain a trained amino acid sequence prediction model.

In some embodiments of this application, the amino acid sequence prediction model is corrected based on the loss result to implement training of the amino acid sequence prediction model, so that the amino acid sequence prediction model of the sample amino acid sequence representation may be predicted accurately, and thus, the antigenic specificity of the cell receptor is determined based on the accurately predicted sample amino acid sequence representation.

In some embodiments, upon training the amino acid sequence prediction model to obtain the trained amino acid sequence prediction model, the amino acid sequence prediction model may further be fine-tuned.

In some embodiments of this application, a multilayer perceptron may be connected behind the amino acid sequence prediction model. The multilayer perceptron is configured to determine the antigenic specificity of the cell receptor. The model fine tuning may be performed on the trained amino acid sequence prediction model and the multilayer perceptron based on the epitope information of the sample cell receptor, and the trained amino acid sequence prediction model and the model parameters in the multilayer perceptron are fine-tuned only.

In the process of fine-tuning the trained amino acid sequence prediction model, fine-tuned sample data may be acquired, where the fine-tuned sample data includes unmasked double-stranded sample biological information and epitope information of the sample cell receptor. Here, the unmasked double-stranded sample biological information is the double-stranded sample biological information of the sample cell receptor. Then, the model parameters in the trained amino acid sequence prediction model are fine-tuned with the unmasked double-stranded sample biological information by taking the epitope information as label data to obtain a fine-tuned amino acid sequence prediction model. Upon obtaining the fine-tuned amino acid sequence prediction model, in a case that feature extraction is performed on the cell receptor in the process of determining the antigenic specificity, feature extraction on the cell receptor may be performed with the fine-tuned amino acid sequence prediction model to obtain the amino acid sequence representation of the cell receptor. That is to say, the unmasked double-stranded sample biological information and the epitope information (the epitope information is used as the label data) of the correspondingly recognized sample cell receptor are inputted into the trained amino acid sequence prediction model to further finely tune the trained amino acid sequence prediction model, so that the fine-tuned amino acid sequence prediction model is capable of performing feature extraction on the cell receptor.

In the process of fine-tuning the multilayer perceptron, fine-tuned sample data may be acquired, where the fine-tuned sample data includes unmasked double-stranded sample biological information and epitope information of the sample cell receptor. Here, the unmasked double-stranded sample biological information is the double-stranded sample biological information of the sample cell receptor. Then, the model parameters in the multilayer perceptron are fine-tuned with the unmasked double-stranded sample biological information by taking the epitope information as the label data to obtain a fine-tuned multilayer perceptron. Upon obtaining the fine-tuned multilayer perceptron, in response to that the antigenic specificity is determined, the antigenic specificity may be determined with the fine-turned multilayer perceptron. The amino acid sequence representation of the fine-tuned amino acid sequence prediction model and the epitope information (the epitope information is used as the label data) of the correspondingly recognized sample cell receptor are inputted into the multilayer perceptron to further finely tune the trained multilayer perceptron, so that the fine-tuned multilayer perceptron is capable of performing accurate recognition on the antigenic specificity of the cell receptor.

In some embodiments of this application, to finely tune the model parameters in the trained amino acid sequence prediction model and the multilayer perceptron may, on the one hand, make the trained amino acid sequence prediction model be capable of being more adapted to the demand of an antigenic specificity recognition task, and on the other hand, achieve training of the multilayer perceptron.

In some embodiments of this application, upon fine-tuning the model parameters in the trained amino acid sequence prediction model and the multilayer perceptron, the fine-tuned amino acid sequence prediction model and the multilayer perceptron are obtained. In this case, feature extraction may be performed on the cell receptor with the fine-tuned amino acid sequence prediction model, and the antigenic specificity of the cell receptor may be determined with the fine-tuned multilayer perceptron. That is to say, the step of performing feature extraction on the cell receptor based on the amino acid word sequence to obtain the amino acid sequence representation of the cell receptor may be implemented with the fine-tuned amino acid sequence prediction model. The step of determining the antigenic specificity of the cell receptor based on the amino acid sequence representation may be implemented with the fine-tuned multilayer perceptron.

The epitope information is explained below.

The epitope information refers to the true information of the sample cell receptor, and model fine tuning is performed on the epitope information as the label information in the sample data. The epitope information includes an epitope of the sample cell receptor. The epitope is a chemical group existing on the surface of an antigen to determine a special structure of the antigenic specificity, which is also called an antigenic determinant. The antigen binds with a surface antigen receptor of a corresponding lymphocyte to activate the lymphocyte, to induce immune response; and the antigen also binds specifically with the corresponding antibody or sensitized lymphocyte. A single antigen molecule may have one or more different epitopes, the epitopes are equivalent to antigen binding sites of the corresponding antibody in size, and each epitope has only one antigenic specificity. Because the epitopes in the epitope information are determined, the true antigenic specificity of the sample cell receptor may be determined based on the epitope information.

Based on the method for training the amino acid sequence prediction model, some embodiments of this application further provide a data preprocessing method for performing data preprocessing on the acquired pre-trained data. FIG. 6 is a schematic diagram of an implementation flow chart of the data preprocessing method provided in an embodiment of this application. As shown in FIG. 6, the method includes the following steps S601 to S604:

Step S601: Data belonging to a specified object is screened out from the pre-trained data.

Here, the pre-trained data is data acquired from a database from different objects. A target object needed to be screened may be preset as a specific object. For example, in a case that a human immune system is to be analyzed, human data may be screened out.

Step S602: Double-stranded data pair analysis is performed on the data belonging to the specified object to obtain a plurality of pieces of double-stranded paired data, where the double-stranded paired data refers to data of the two paired peptide chains paired with each other.

Here, the double-stranded paired data analysis means that whether the data belonging to the specific object has the data of two peptide chains capable of being paired is analyzed. In response to that any one piece of data has the data of the two peptide chains capable of being paired, the data is determined as the double-stranded paired data; and in response to that any one piece of data does not have the data of the two peptide chains capable of being paired or only has single-stranded data, the data may be eliminated from the data belonging to the specific object.

Step S603: Data length analysis is performed on each piece of the double-stranded paired data to obtain a data length of the double-stranded paired data.

Here, the data length refers to the quantity of the amino acids in the amino acid sequence corresponding to the double-stranded paired data. For example, in a case that the quantity of the amino acids in the amino acid sequence (i.e., the double-stranded paired data) corresponding to the two paired peptide chains of any specific object is 40, the data length of the double-stranded paired data is 40.

Step S604: The double-stranded paired data with data length less than a length threshold is determined as the sample data.

Here, the double-stranded paired data with the data length greater than or equal to the length threshold is eliminated to obtain the sample data. Because the CDR3 sequence of the specific object has a certain length range, in a case that the data length of any double-stranded paired data exceeds the length range, it indicates that the double-stranded paired data has data exception or does not have the universality of model learning, and thus, the double-stranded paired data may be eliminated.

For example, the length of a CDR3 region is usually less than 40, and for the data in a pre-training stage and a downstream classification stage, a CDR3 sequence with the length greater than 40 may be screened out.

In some embodiments, data elimination processing may also be performed on the epitope information in the sample data, an epitope length threshold may be preset, and the epitope length corresponding to the epitope information is determined. Here, the epitope length refers to the quantity of the amino acids in antigen epitope sequence corresponding to the epitope information. The epitope information with the epitope length greater than or equal to the epitope length threshold may be deleted, i.e., the antigen epitope sequences with the epitope length greater than or equal to the epitope length threshold are deleted.

In some embodiments of this application, specific object recognition and screening, double-stranded data pairing analysis and screening and length analysis and screening are performed successively on the pre-trained data acquired from the database to obtain the sample data capable of accurately training the amino acid sequence prediction model.

Description of an embodiment of the application in a use scenario will be made below.

The human immune system includes congenital immunity and adaptive immunity. Adaptive immunity is an immune response which generates and targets initiation of a specific pathogen via contact with the specific pathogen (antigen). A T cell and a B cell are significant components for an adaptive immune system. Antigen recognition is one of the critical factors for T cell and B cell mediated immunity. The T cell and the B cell are mainly formed by interactions between a T cell receptor (TCR, a protein dimer) and a B cell receptor (BCR) and the antigen. The TCR recognizes and binds with the antigen present on a major histocompatibility complex (MHC) on a cell membrane and the BCR directly binds with the specific antigen. The TCR and the BCR each have two peptide chains (an α chain and a B chain or a heavy chain or a light chain). The two chains form an annular three-dimensional structure (including CDR1, CDR2 and CDR3) of a complementarity determining region (CDR) for antigen recognition and binding. In a case that the TCR recognizes an MHC-antigen peptide complex, the CDR1 and CDR2 recognize and bind with a side wall of an MHC molecular antigen binding cleft and the CDR3 directly binds with the antigen peptide; and variation in CDR3 is the maximum, which directly decides the antigen binding specificity of the TCR/BCR. Predicting the antigenic specificity of TCR/BCR based on the TCR/BCR sequence information and gene information and accurately predicting the activating capacity of the T cell/B cell based on the antigenic specificity have a transformative impact on many leading research fields such as treatment of infectious diseases and autoimmune diseases and design of immune vaccines for cancers. The difficulties to predict the antigenic specificity of the TCR/BCR are as follows: first, from the biological perspective, because genes are randomly recombined, many types of TCR/BCR may be generated theoretically; and second, from the perspective of machine learning training, data with known antigenic specificity capable of being collected is less. Owning to less data with labels and the sequence data used, a “protein language” may be learned in a supervised manner with the BERT network of natural language processing to obtain codes characterizing the TCR/BCR, to predict the downstream specificity of the TCR/BCR.

Some embodiments of this application provide a method for predicting antigenic specificity of an immune receptor based on BERT, which predicts the antigenic specificity of the TCR/BCR with amino acid sequence information.

Prediction of the antigenic specificity of the TCR/BCR receptor is a core problem in immunology. predicting the antigenic specificity of TCR/BCR based on the TCR/BCR sequence information and gene information and accurately predicting the activating capacity of the T cell/B cell based on the antigenic specificity have a transformative impact on many leading research fields such as treatment of infectious diseases and autoimmune diseases and design of immune vaccines for cancers.

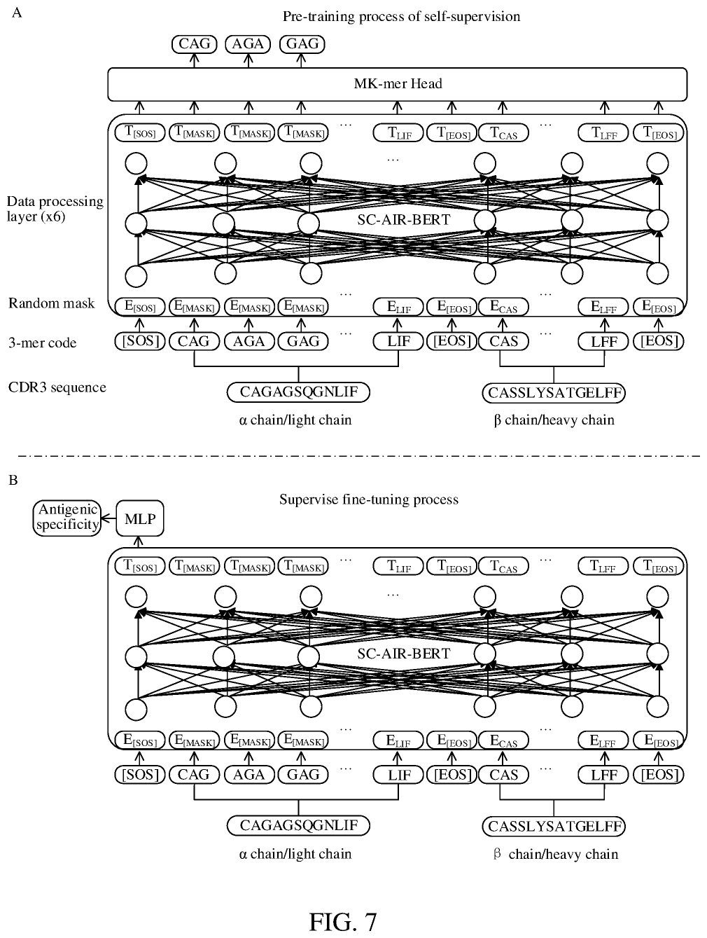

FIG. 7 is a schematic diagram of a model framework of an algorithm corresponding to the method for determining antigenic specificity provided in some embodiments of this application. As shown in FIG. 7, in an implementation process, the CDR3 sequence of the two peptide chains are respectively processed into kmer sequences, each kmer representing a word, and the whole kmer sequence serves as a sentence. Marking, segmenting and location coding are performed on the two connected sentences (because the BERT is a language model, one CDR3 sequence is naturally taken as a sentence here. But there is no clear concept of words in a biological sequence, 3 amino acids may be selected as a word by comprehensively considering the length of the CDR3 sequence; two amino acids are overlapping between two adjacent words).

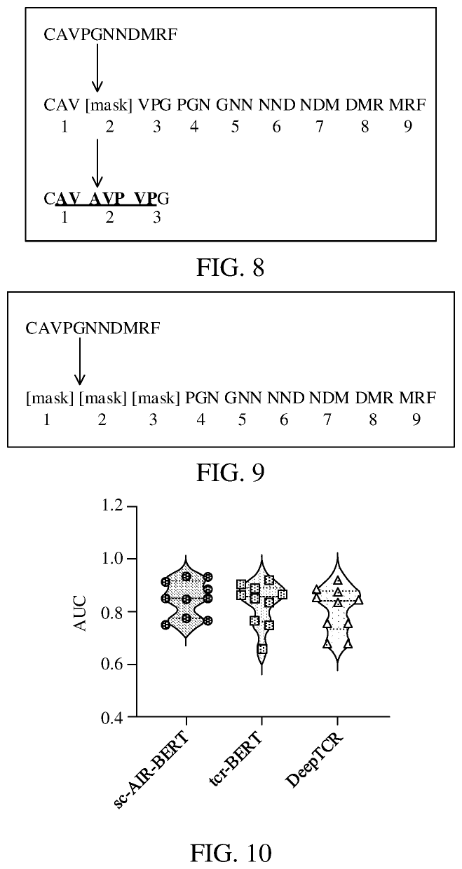

Continuously referring the portion A in FIG. 7, the coded TCR/BCR is inputted into a BERT model of a six-layered data processing layer; and the BERT model is pre-trained with large data without the epitope information (without labels). The protein language in the TCR/BCR is pre-learned into a model by means of the self-supervising technology to extract some universal features in the TCR/BCR sequence, to acquire the codes of the CDR3 sequence. The BERT network may be modified as follows: because the two peptide chains of the TCR/BCR area paired relatively randomly, an SNP task of the BERT model is removed with only an MLM task reserved. At the same time, a mask strategy is modified: Because the kmer sequence is used as the input data, there will be two overlapping amino acids between words, and therefore, in a case that a certain kmer is masked, two words on the left and right sides of kmer both are masked. As shown in FIG. 8, it is a schematic diagram of a masking effect before the mask strategy is modified. During the mask processing, in a case that the second word “AVP” is to be masked and only the second word “AVP” is masked, the model will speculate the kmer easily through the words on the left and right sides, for example, the second word is speculated to be “AVP” through “AV” in the first word “CAV” and “VP” in the first word “VPG”. Therefore, during the mask processing, in a case that the second word “AVP” is masked, it is needed to mask “AV” in the first word “CAV”, and moreover, it is also needed to mask “VP” in the third word “VGP”. FIG. 9 is a schematic diagram of a masking effect after the mask strategy is modified. It may be seen that to prevent the model from speculating the kmer easily from the words on the left and right sides, as shown in FIG. 9, the first word and the third word adjacent to the second word are partially masked, which is capable of preventing model training from being converged too fast. In the mask processing process shown in FIG. 9, two amino acids behind the first word are masked, and two amino acids in front of the third word are masked.

In some embodiments of this application, the BERT model may be fine-tuned with the data with the epitope information, as shown in the portion B in FIG. 7, and the learned multimodal feature is mapped to the final prediction result of the antigenic specificity of the TCR/BCR as one classification head includes a multilayer perceptron.

The data preprocessing flow of the algorithm corresponding to the method for determining antigenic specificity provided in some embodiments of this application is described below.

For the pre-trained data, a pre-trained data table of TCR/BCR shown in FIG. 1, data of two databases: VDJDB and IEDB, and three data sets: 10×PBMC, nasopharyngeal carcinoma (NPC) and inflammatory bowel disease (IBD) are collected. The data in a pre-training task is processed as follows: only human data is collected, and it is required that the data is paired with the CDR3 sequence of the α chin and β chain; and the data with the length greater than 40 is also eliminated, including the data of the TCR and the BCR. This is because on the one hand, the CDR3 sequence of the TCR and the BCR has some universal rules to learn; and on the other hand, it is expected that the available range of the pre-trained model may be wider (may be applied to TCR and BCR tasks). For the data in the pre-training stage and the downstream classification stage, the CDR3 sequence with the length greater than 40 may be screened out (the length of the CDR3 region is usually less than 40). For the data in the downstream classification stage, the antigen epitope sequence with the length greater than 50 is screened out. For the two data sets of the downstream classification task, the 10×PBMC data set is from 4 denotation values, and quality control (QC) is made on the data collected from a 10× official website. Data including 7 epitopes are screened, including the downstream classification data −10×PBMC shown in FIG. 2. The VDJ data set includes data including three epitopes collected from vdjdb; and the data on vdjdb is usually data in a published article, which is reliable, for example, the downstream classification data-VDJ shown in table 3. (1049/5562 in table 3 represents that the number of the TCR binding with epitope GILGFVFTL is 1049 and the total number of the TCR is 5562)

TABLE 1

| Data set | TCR/BCR | | Data size |
|---|---|---|---|
| VDJDB | T | | 23358 |
| IEDB | T | | 18662 |
| | B | | 224 |
| 10xPMBC | T | | 5834 |
| NPC | T | | 13400 |
| | B | | 14647 |
| IBD | T | Peripheral blood mononuclear cell | 36108 |
| | | Rectal immune cell | 29182 |
| | | Ileal immune cell | 7363 |
| | B | Peripheral blood mononuclear cell | 9358 |
| | | Rectal immune cell | 19430 |
| | | Ileal immune cell | 7164 |

| | | |
|---|---|---|
| Total | — | 163486 |

TABLE 2

| Epitope | GILGFVFTL | KLGGALQAK | RAKFKQLL | AVFDRKSDAK | GLCTLVAML |
|---|---|---|---|---|---|
| TCR data | 1049/5562 | 2917/5562 | 923/5562 | 121/5562 | 93/5562 |
| size | | | | | |
| | | | Epitope | IVTDFSVIK | ELAGIGILTV |
| | | | TCR data | 250/5562 | 209/5562 |
| | | | size | | |

TABLE 3

| Epitope | LTDEMIAQY | TTDPSFLGRY | YLQPRTFLL |
|---|---|---|---|
| TCR data size | 132/592 | 254/592 | 206/592 |

Upon receiving the pre-trained data, the TCR/BCR sequence is processed into the kmer sequence. BERT is the language model; one CDR3 sequence may be naturally taken as a sentence. But there is no clear concept of words in the biological sequence, and 3 amino acids may be selected as a word by comprehensively considering the length of the CDR3 sequence with reference to the preceding content; and two amino acids are overlapping between two adjacent words.

In the model training stage, the model (i.e., the amino acid sequence prediction model, a model provided based on the BERT technology represented as sc-AIR-BERT) is trained in an end-to-end manner by using the cross entropy function as the loss function. In the task prediction stage, the model trained may directly predict the antigenic specificity recognition problem of the TCR/BCR.

Compared with other methods in terms of the public data set in some embodiments of this application, performance comparison on antigenic specificity recognition of the TCR is shown in FIG. 10, where the sc-AIR-BERT is the method provided in some embodiments of this application. It may be seen that the AUC value of a predicted result of the sc-AIR-BERT model provided in some embodiments of this application is 0.835 which is greater than the AUC values of the predicted results of trc-BERT and DeepTCR models. Here, the AUC here refers to a probability that by randomly giving a positive sample and a negative sample, the probability value that the classifier outputs the positive sample positively is greater than the probability that the classifier outputs the negative sample positively.

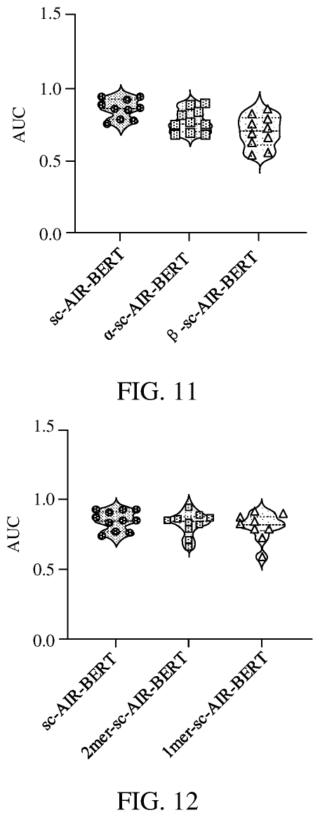

FIG. 11 is a schematic diagram of performance comparison of an embodiment of this application on the public data set and a single-stranded method on TCR antigenic specificity recognition. As shown in FIG. 11, α shows the performance of the method in some embodiments of this application in a case that only α chain data is used, and β shows the performance of the method in some embodiments of this application in a case that only β chain data is used. It may be seen that the AUC value of the predicted value of the model in a case that both the a chain data and the β chain data are used is greater than the performance in a case that only the α chain data is used and the performance in a case that only the ß chain data is used.

FIG. 12 is a schematic diagram of performance comparison of an embodiment of this application on the public data set and a single-stranded method on TCR antigenic specificity recognition. As shown in FIG. 12, 2mer-BERT is the performance in a case that 2mer data is used in some embodiments of this application, and 1mer-BERT is the performance in a case that 1mer data is used in some embodiments of this application. It may be seen that the AUC value corresponding to the performance (i.e., sc-AIR-BERT) in a case that 3mer data is used is greater than the performance of 2mer-sc-AIR-BERT and 1mer-sc-AIR-BERT, that is to say, the performance in a case that the 3mer data is used is higher than the performance in a case that the 2mer data is used and the performance in a case that the 1mer data is used.

The method for determining the antigenic specificity provided in some embodiments of this application introduces the TCR/BCR sequences paired by BERT pre-training for analysis of adaptive immune receptors and learns the “protein language” in a self-supervising manner by means of the BERT network to acquire codes of the paired TCR/BCR sequences. Implementing the embodiments of this application provide many leading research fields such as treatment of infectious diseases and autoimmune diseases and design of immune vaccines for cancers with a technical support.

The pre-trained model in some embodiments of this application is capable of characterizing the amino acid sequence as codes. The pre-training model is applied to predicting TCR/BCR specificity and may further be used in other downstream tasks such as prediction of TCR/BCR affinity and disease prediction. The model in some embodiments of this application may further be fused with V(D)J gene information for a subsequent prediction task. Besides kmer coding, the coding modes may be other codes in a case that data preprocessing is performed, including, but not limited to, Achely factor, kidera factor and one-hot coding.

It may be understood that in the embodiments of this application, in a case that the contents involving user information such as double-stranded biological information and antigenic specificity and disease related information refer to data related to the user information or enterprise information, in a case that the embodiments of this application are applied to specific products or technologies, it is needed to obtain user permission or consent, and collection, use and processing of related data need to comply with relevant laws and regulations and standards of relevant countries and regions.

An apparatus 354 for determining antigenic specificity provided in some embodiments of this application is implemented as a software module is continuously described below. In some embodiments, as shown in FIG. 2, the apparatus 354 for determining antigenic specificity includes:

an acquisition module, configured to acquire double-stranded biological information of a cell receptor; a word coding module, configured to perform word coding processing on the double-stranded biological information of the cell receptor to obtain an amino acid word sequence, the amino acid word sequence including at least one amino acid word representation; a feature extraction module, configured to perform feature extraction on the cell receptor based on the amino acid word sequence with a pre-trained amino acid sequence prediction model, the amino acid prediction model being trained with masked sample data, and the masked sample data being data obtained by mask processing of a part of sample amino acid word representations in sample data; and a determination module, configured to determine the antigenic specificity of the cell receptor based on the amino acid sequence representations.

In some embodiments, the word coding module is further configured to: determine monomeric unit quantities corresponding to the word coding processing; and code each monomeric unit quantity of continuous amino acids in the double-stranded biological information of the cell receptor into an amino acid word representation, where the double-stranded biological information is coded to obtain a plurality of amino acid word representations to form the amino acid word sequence; and there is a preset quantity of overlapping amino acids between two adjacent amino acid word representations, and the preset quantity is 1 less than the monomeric unit quantity.

In some embodiments, the acquisition module is further configured to: acquire two peptide chains of the cell receptor; and determine the double-stranded biological information of the cell receptor based on the two peptide chains.

In some embodiments, the cell receptor includes a T cell receptor or a B cell receptor; in a case that the cell receptor is the T cell receptor, the two peptide chains include an α chain and a β chain; in a case that the cell receptor is the B cell receptor, the two peptide chains include a heavy chain and a light chain; and a value of the monomeric unit quantity is 3.

In some embodiments, the double-stranded biological information includes amino acid information corresponding to each of the peptide chains in the two peptide chains; the word coding module is further configured to: code each monomeric unit quantity of continuous amino acids in each of the peptide chains into an amino acid word representation, where the plurality of amino acid word representations in each of the peptide chains form an amino acid subsequence corresponding to the peptide chain; and determine the amino acid word sequence according to the amino acid subsequences corresponding to the two peptide chains.

In some embodiments, the word coding module is further configured to: splice the amino acid subsequences corresponding to the two peptide chains to obtain a spliced word sequence; mark the spliced word sequence to obtain a marked spliced word sequence; segment the marked spliced word sequence to obtain a segmented spliced word sequence; and perform location coding processing on the segmented spliced word sequence to obtain the amino acid word sequence.

In some embodiments, the apparatus further includes: a model training module, configured to train the amino acid sequence prediction model, where the model training module is further configured to: perform data preprocessing on the acquired pre-trained data to obtain sample data, where the sample data includes sample double-stranded biological information of the sample cell receptor; input the sample double-stranded biological information into the amino acid sequence prediction model; perform word coding processing on the sample double-stranded biological information through a word coding processing layer of the amino acid sequence prediction model to obtain a sample amino acid word sequence, where the sample amino acid word sequence includes at least one sample amino acid word representation; perform mask processing on the at least one sample amino acid word representation in the sample amino acid word sequence through a mask processing layer of the amino acid sequence prediction model to obtain a masked sample amino acid word sequence; predict the amino acid sequence of the sample cell receptor based on the masked sample amino acid word sequence through a prediction processing layer of the amino acid sequence prediction model to obtain a masked sample amino acid word representation during the mask processing, and determine a sample amino acid sequence representation of the sample cell receptor based on the predicted masked sample amino acid word representation; input the sample amino acid sequence representation into a preset loss model to obtain a loss result; and correct model parameters in the word coding processing layer, the mask processing layer and the prediction processing layer based on the loss result to obtain a trained amino acid sequence prediction model.

In some embodiments, the feature extraction module is further configured to: acquire fine-tuned sample data, where the fine-tuned sample data includes unmasked double-stranded sample biological information and epitope information of the sample cell receptor; finely tune the model parameters in the trained amino acid sequence prediction model with the unmasked double-stranded sample biological information by taking the epitope information as label data to obtain a fine-tuned amino acid sequence prediction model; and perform feature extraction on the cell receptor with the fine-tuned amino acid sequence prediction model to obtain the amino acid sequence representation of the cell receptor.

In some embodiments, a process of determining the antigenic specificity of the cell receptor is achieved by a multilayer perceptron; and the determination module is further configured to: finely tune the model parameters in the multilayer perceptron with the unmasked double-stranded sample biological information by taking the epitope information as label data to obtain a fine-tuned multilayer perceptron; and determine the antigenic specificity of the cell receptor with the fine-tuned multilayer perceptron based on the amino acid sequence representations.

In some embodiments, the model training module is further configured to: determine at least one sample amino acid word representation randomly selected as a target amino acid word representation from the sample amino acid word sequence; determine adjacent amino acid word representations adjacent to the target amino acid word representation, where the adjacent amino acid word representations include: a first adjacent amino acid word representation adjacent to a first side of the target amino acid word representation and a second adjacent amino acid word representation adjacent to a second side of the target amino acid word representation; in a case that the target amino acid word representation is located at a sequence starting location of the amino acid word sequence, the adjacent amino acid word representations include a second adjacent amino acid word representation adjacent to the second side of the target amino acid word representation; and in a case that the target amino acid word representation is located at a sequence end location of the amino acid word sequence, the adjacent amino acid word representations include a first adjacent amino acid word representation adjacent to the first side of the target amino acid word representation; and perform mask processing on overlapping amino acids in the target amino acid word representation and the first adjacent amino acid word representation and overlapping amino acids in the second adjacent amino acid word representation to obtain the masked sample amino acid word sequence.

In some embodiments, the model training module is further configured to: input the sample amino acid sequence representation and the sample double-stranded biological information into the preset loss model; determine a sequence distance between the sample amino acid sequence representation and the sample double-stranded biological information through a cross entropy loss function in the preset loss model; and determine the loss result according to the sequence distance.

In some embodiments, the amino acid sequence representation is a multimodal feature; and the determination module is further configured to: input the amino acid sequence representation into the fine-tuned multilayer perceptron; and perform mapping processing on the multimodal feature corresponding to the amino acid sequence representation through the fine-tuned multilayer perceptron to obtain the antigenic specificity of the cell receptor.

In some embodiments, the model training module is further configured to: filter out data belonging to a specified object from the pre-trained data; perform double-stranded data pair analysis on the data belonging to the specified object to obtain a plurality of pieces of double-stranded paired data, where the double-stranded paired data refers to data of the two paired peptide chains paired with each other; perform data length analysis on each piece of the double-stranded paired data to obtain a data length of the double-stranded paired data; and determine the double-stranded paired data with the data length less than a length threshold as the sample data.

The descriptions of the embodiment apparatus of this application are similar to the foregoing descriptions of the method embodiment, and the apparatus embodiment has beneficial effects similar to those of the method embodiment, and therefore is not described in detail. Refer to descriptions in the method embodiment of this application for technical details undisclosed in the apparatus embodiment of this application.

Some embodiments of this application provide a computer program product or a computer program, the computer program product or the computer program including an executable instruction, the executable instruction being a computer instruction; and the executable instruction being stored in a computer readable storage medium. The processor of the electronic device reads the executable instruction from the computer readable storage medium, causing the electronic device to execute the method in some embodiments of this application when executing the executable instruction.

Some embodiments of this application provides a storage medium storing the executable instruction, storing the executable instruction, and causing the processor to execute the method provided in some embodiments of this application when executing the executable instruction by the processor, for example, the method show in FIG. 3.

In some embodiments, the storage medium may be a computer-readable storage medium, for example, memories such as a ferromagnetic random access memory (FRAM), a read-only memory (ROM), a programmable read-only memory (PROM), an erasable programmable read-only memory (EPROM), an electrically erasable programmable read-only memory (EEPROM), a flash memory, a magnetic surface memory, an optic disc, or a compact disc read-only memory (CD-ROM). The storage medium may also be various devices including one of or any combination of the above memories.

In some embodiments, the executable instruction may be in the form of program, software, software module, script or code, which is compiled by any form programming language (including a compiling language or interpretive language or a declarative language or procedural language). Moreover, the executable instruction may be deployed in any form including an independent program or a module, a component, a subroutine or other units suitable for use in a computing environment.

As an example, the executable instruction may, but not necessarily, correspond to a file in a file system, and may be stored in one or more scripts in a hypertext markup language (HTML) document, a single file dedicated for the discussed program or a plurality of cooperative files (for example, a file storing one or more modules, subprograms or codes) as a part capable of being stored in a file saving other programs or data. An as example, the executable instruction may be deployed on an electronic device to execute or may be executed by a plurality of electronic devices located on a same location or may be executed by a plurality of electronic devices distributed at a plurality of locations and interconnected via a communication network.

The foregoing descriptions are merely preferred embodiments of this application and are not intended to limit the protection scope of this application. Any modification, equivalent replacement, or improvement made without departing from the spirit and range of this application shall fall within the protection scope of this application.

What is claimed is:

1. A method for determining antigenic specificity, executed by an electronic device, the method comprising:
acquiring double-stranded biological information of a cell receptor;
performing word coding processing on the double-stranded biological information to obtain an amino acid word sequence, the amino acid word sequence comprising an amino acid word representation;
performing feature extraction on the cell receptor based on the amino acid word sequence with a pre-trained amino acid sequence prediction model to obtain an amino acid sequence representation of the cell receptor, the amino acid prediction model being trained with masked sample data; and
determining the antigenic specificity of the cell receptor based on the amino acid sequence representations.

2. The method according to claim 1, wherein the operation of performing word coding processing on double-stranded biological information of a cell receptor to obtain an amino acid word sequence comprises:
determining monomeric unit quantities corresponding to the word coding processing as N; and
coding each N continuous amino acids in the double-stranded biological information of the cell receptor into an amino acid word representation, wherein the double-stranded biological information is coded to obtain a plurality of amino acid word representations to form the amino acid word sequence, and two adjacent amino acid word representations having N−1 overlapping amino acids.

3. The method according to claim 2, wherein acquiring double-stranded biological information of a cell receptor comprises:
acquiring two peptide chains of the cell receptor; and
determining the double-stranded biological information of the cell receptor based on the two peptide chains.

4. The method according to claim 3, wherein the cell receptor comprises a T cell receptor or a B cell receptor; in a case that the cell receptor is the T cell receptor, the two peptide chains comprise an α chain and a β chain; in a case that the cell receptor is the B cell receptor, the two peptide chains comprise a heavy chain and a light chain; and
a value of N is 3.

5. The method according to claim 3, wherein the double-stranded biological information comprises amino acid information corresponding to each of the peptide chains in the two peptide chains; and
the operation of coding each N continuous amino acids in the double-stranded biological information of the cell receptor into an amino acid word representation to form the amino acid word sequence comprises:
coding each N continuous amino acids in each of the peptide chains into an amino acid word representation, wherein the plurality of amino acid word representations in each of the peptide chains form an amino acid subsequence corresponding to the peptide chain; and
determining the amino acid word sequence according to the amino acid subsequences corresponding to the two peptide chains.

6. The method according to claim 5, wherein determining the amino acid word sequence according to the amino acid subsequences corresponding to the two peptide chains comprises:
splicing the amino acid subsequences corresponding to the two peptide chains to obtain a spliced word sequence;
marking the spliced word sequence to obtain a marked spliced word sequence;
segmenting the marked spliced word sequence to obtain a segmented spliced word sequence; and
performing location coding processing on the segmented spliced word sequence to obtain the amino acid word sequence.

7. The method according to claim 1, wherein the amino acid sequence prediction model is trained by
performing data preprocessing on the acquired pre-trained data to obtain sample data, wherein the sample data comprises sample double-stranded biological information of a sample cell receptor;
inputting the sample double-stranded biological information into the amino acid sequence prediction model;
performing word coding processing on the sample double-stranded biological information through a word coding processing layer of the amino acid sequence prediction model to obtain a sample amino acid word sequence, wherein the sample amino acid word sequence comprises at least one sample amino acid word representation;
performing mask processing on the at least one sample amino acid word representation in the sample amino acid word sequence through a mask processing layer of the amino acid sequence prediction model to obtain a masked sample amino acid word sequence;
predicting the amino acid sequence of the sample cell receptor based on the masked sample amino acid word sequence through a prediction processing layer of the amino acid sequence prediction model to obtain a masked sample amino acid word representation during the mask processing, and determining a sample amino acid sequence representation of the sample cell receptor based on the predicted masked sample amino acid word representation;
inputting the sample amino acid sequence representation into a preset loss model to obtain a loss result; and
correcting model parameters in the word coding processing layer, the mask processing layer and the prediction processing layer based on the loss result to obtain a trained amino acid sequence prediction model.

8. The method according to claim 7, wherein performing feature extraction on the cell receptor based on the amino acid word sequence with a pre-trained amino acid sequence prediction model to obtain an amino acid sequence representation of the cell receptor comprises:
acquiring fine-tuned sample data, wherein the fine-tuned sample data comprises unmasked double-stranded sample biological information and epitope information of the sample cell receptor;
tuning the model parameters in the trained amino acid sequence prediction model with the unmasked double-stranded sample biological information by taking the epitope information as label data to obtain a fine-tuned amino acid sequence prediction model; and
performing feature extraction on the cell receptor with the fine-tuned amino acid sequence prediction model to obtain the amino acid sequence representation of the cell receptor.

9. The method according to claim 8, wherein determining the antigenic specificity of the cell receptor is achieved by a multilayer perceptron; and the operation of determining the antigenic specificity of the cell receptor based on the amino acid sequence representations comprises:
tuning the model parameters in the multilayer perceptron with the unmasked double-stranded sample biological information by taking the epitope information as label data to obtain a fine-tuned multilayer perceptron; and
determining the antigenic specificity of the cell receptor with the fine-tuned multilayer perceptron based on the amino acid sequence representations.

10. The method according to claim 7, wherein performing mask processing on the at least one sample amino acid word representation in the sample amino acid word sequence to obtain a masked sample amino acid word sequence comprises:
determining at least one sample amino acid word representation randomly selected as a target amino acid word representation from the sample amino acid word sequence;
determining adjacent amino acid word representations adjacent to the target amino acid word representation, wherein the adjacent amino acid word representations comprise: a first adjacent amino acid word representation adjacent to a first side of the target amino acid word representation and a second adjacent amino acid word representation adjacent to a second side of the target amino acid word representation; in a case that the target amino acid word representation is located at a sequence starting location of the amino acid word sequence, the adjacent amino acid word representations comprise a second adjacent amino acid word representation adjacent to the second side of the target amino acid word representation; and in a case that the target amino acid word representation is located at a sequence end location of the amino acid word sequence, the adjacent amino acid word representations comprise a first adjacent amino acid word representation adjacent to the first side of the target amino acid word representation; and
performing mask processing on overlapping amino acids in the target amino acid word representation and the first adjacent amino acid word representation and overlapping amino acids in the second adjacent amino acid word representation to obtain the masked sample amino acid word sequence.

11. The method according to claim 7, wherein inputting the sample amino acid sequence representation and the epitope information into a preset loss model to obtain a loss result comprises:
inputting the sample amino acid sequence representation and the sample double-stranded biological information into the preset loss model;
determining a sequence distance between the sample amino acid sequence representation and the sample double-stranded biological information through a cross entropy loss function in the preset loss model; and
determining the loss result according to the sequence distance.

12. The method according to claim 9, wherein the amino acid sequence representation is a multimodal feature; and determining the antigenic specificity of the cell receptor with the fine-tuned multilayer perceptron based on the amino acid sequence representations comprises:
inputting the amino acid sequence representation into the fine-tuned multilayer perceptron; and
performing mapping processing on the multimodal feature corresponding to the amino acid sequence representation through the fine-tuned multilayer perceptron to obtain the antigenic specificity of the cell receptor.

13. The method according to claim 7, wherein performing data preprocessing on the acquired pre-trained data to obtain sample data comprises:
screening data belonging to a specified object from the pre-trained data;
performing double-stranded data pair analysis on the data belonging to the specified object to obtain a plurality of pieces of double-stranded paired data, wherein the double-stranded paired data refers to data of the two paired peptide chains paired with each other;
performing data length analysis on each piece of the double-stranded paired data to obtain a data length of the double-stranded paired data; and
determining the double-stranded paired data with the data length less than a length threshold as the sample data.

14. An electronic device, comprising:
a memory, configured to store executable instructions; and a processor, configured to implement a method for determining antigenic specificity when executing the executable instructions stored in the memory, the method comprising:
acquiring double-stranded biological information of a cell receptor;
performing word coding processing on the double-stranded biological information to obtain an amino acid word sequence, the amino acid word sequence comprising an amino acid word representation;
performing feature extraction on the cell receptor based on the amino acid word sequence with a pre-trained amino acid sequence prediction model to obtain an amino acid sequence representation of the cell receptor, the amino acid prediction model being trained with masked sample data; and
determining the antigenic specificity of the cell receptor based on the amino acid sequence representations.

15. The electronic device according to claim 14, wherein the operation of performing word coding processing on double-stranded biological information of a cell receptor to obtain an amino acid word sequence comprises:
determining monomeric unit quantities corresponding to the word coding processing as N; and
coding each N continuous amino acids in the double-stranded biological information of the cell receptor into an amino acid word representation, wherein the double-stranded biological information is coded to obtain a plurality of amino acid word representations to form the amino acid word sequence, and two adjacent amino acid word representations having N−1 overlapping amino acids.

16. The electronic device according to claim 15, wherein acquiring double-stranded biological information of a cell receptor comprises:
acquiring two peptide chains of the cell receptor; and
determining the double-stranded biological information of the cell receptor based on the two peptide chains.

17. The electronic device according to claim 16, wherein the cell receptor comprises a T cell receptor or a B cell receptor; in a case that the cell receptor is the T cell receptor, the two peptide chains comprise an α chain and a β chain; in a case that the cell receptor is the B cell receptor, the two peptide chains comprise a heavy chain and a light chain; and
a value of N is 3.

18. A non-transitory computer readable storage medium, storing the executable instructions, and configured to cause a processor to implement a method for determining antigenic when executing the executable instructions, the method comprising:
acquiring double-stranded biological information of a cell receptor;
performing word coding processing on the double-stranded biological information to obtain an amino acid word sequence, the amino acid word sequence comprising an amino acid word representation;
performing feature extraction on the cell receptor based on the amino acid word sequence with a pre-trained amino acid sequence prediction model to obtain an amino acid sequence representation of the cell receptor, the amino acid prediction model being trained with masked sample data; and
determining the antigenic specificity of the cell receptor based on the amino acid sequence representations.

19. The computer readable storage medium according to claim 18, wherein the operation of performing word coding processing on double-stranded biological information of a cell receptor to obtain an amino acid word sequence comprises:
determining monomeric unit quantities corresponding to the word coding processing as N; and
coding each N continuous amino acids in the double-stranded biological information of the cell receptor into an amino acid word representation, wherein the double-stranded biological information is coded to obtain a plurality of amino acid word representations to form the amino acid word sequence, and two adjacent amino acid word representations having N−1 overlapping amino acids.

20. The computer readable storage medium according to claim 19, wherein acquiring double-stranded biological information of a cell receptor comprises:
acquiring two peptide chains of the cell receptor; and
determining the double-stranded biological information of the cell receptor based on the two peptide chains.
